(19) United States
(12) Patent Application Publication
(10) Pub. No.: US 2025/0375158 A1
(43) Pub. Date: Dec. 11, 2025

(54) SEARCH AND RESCUE ASSISTANCE HARDWARE AND REMOTE MONITORING PLATFORM

(71) Applicants: Kouta Ueki, Amherst, MA (US); Aubin Kevrekidis, Amherst, MA (US); William Larson, Amherst, MA (US); James Hall, Amherst, MA (US); Elizabeth Guider Smith, Amherst, MA (US); Mihir Shenoy, Amherst, MA (US); Yatharth Rajakumar, Amherst, MA (US); Cameron Baird, Amherst, MA (US); Parmida Arghavani, Amherst, MA (US); Rymas Aladmawy, Amherst, MA (US); Max Costa, Amherst, MA (US); Yoh Morita, Amherst, MA (US); Cynthia Tian, Amherst, MA (US); John A. Fabel, Amherst, MA (US); Sam Woodruff, Amherst, MA (US); Rowan G. Reichard, Amherst, MA (US); Alex Lagrant, Amherst, MA (US); Olivia Hansen Fisher, Amherst, MA (US); Kieran Palmer, Amherst, MA (US); Evelyn Ross, Amherst, MA (US)

(72) Inventors: Kouta Ueki, Amherst, MA (US); Aubin Kevrekidis, Amherst, MA (US); William Larson, Amherst, MA (US); James Hall, Amherst, MA (US); Elizabeth Guider Smith, Amherst, MA (US); Mihir Shenoy, Amherst, MA (US); Yatharth Rajakumar, Amherst, MA (US); Cameron Baird, Amherst, MA (US); Parmida Arghavani, Amherst, MA (US); Rymas Aladmawy, Amherst, MA (US); Max Costa, Amherst, MA (US); Yoh Morita, Amherst, MA (US); Cynthia Tian, Amherst, MA (US); John A. Fabel, Amherst, MA (US); Sam Woodruff, Amherst, MA (US); Rowan G. Reichard, Amherst, MA (US); Alex Lagrant, Amherst, MA (US); Olivia Hansen Fisher, Amherst, MA (US); Kieran Palmer, Amherst, MA (US); Evelyn Ross, Amherst, MA (US)

(21) Appl. No.: 19/233,051

(22) Filed: Jun. 10, 2025

Related U.S. Application Data
(60) Provisional application No. 63/658,579, filed on Jun. 11, 2024.

(51) Int. Cl. A61B 5/00; A41D 1/00; A41D 27/20; A61B 5/0205; A61B 5/11; A61B 5/16

(52) U.S. Cl. CPC A61B 5/4875; A41D 1/005; A41D 27/205; A61B 5/0022; A61B 5/02055; A61B 5/1112; A61B 5/165; A61B 5/6805; A61B 5/742; A41D 2500/00; A61B 2560/0214; A61B 2562/0276

(57) ABSTRACT

A garment can include a pocket to hold a removable electronic device. The electronic device can sense body core temperature, heart rate, skin conductivity, blood oxygen level, and geo-spatial location of the wearer, and transmit this data wirelessly to a mapping application. A viewer of the application can track the location and the health status of the wearer. A leader at a search and rescue command post can easily monitor both the location and well-being of personnel through the mapping application while they are on a mission. An algorithm on the backend can determine a health status color based on the sensor data. The pocket design facilitates insertion and removal of the electronics, and ensures that sensors are positioned at optimal locations on the body.

### PRIORITY APPLICATION

This application is a non-provisional application claiming priority to and/or receiving benefit of U.S. Provisional Application No. 63/658,579, titled “SEARCH AND RESCUE ASSISTANCE HARDWARE AND REMOTE MONITORING PLATFORM”, and filed on 11 Jun. 2024. The U.S. Provisional Application is hereby incorporated by reference in its entirety.

### TECHNICAL FIELD

The present disclosure generally relates to search and rescue (SAR) technology, and, more specifically, to search and rescue assistance hardware and a remote monitoring platform.

### BACKGROUND

During emergency situations and disaster scenarios, search and rescue teams are often deployed to remote or hazardous environments to locate and recover missing persons, vehicles, or other targets. The work of these personnel is extremely demanding, requiring extended periods of strenuous physical exertion under harsh conditions that can include extreme temperatures, difficult terrain, limited resources, and associated risks to their health and safety.

Systems for monitoring the status of search and rescue workers have been limited. Voice communications allow periodic check-ins but provide little insight into an individual's physical condition between exchanges. Direct observation is difficult when team members operate over wide areas or in visually-obstructed settings like dense forests or urban wreckage. As a result, injuries, illness, or incapacitation of personnel can go unnoticed until a crisis point, delaying treatment and potentially compounding the emergency.

Similarly, tracking the locations of multiple search and rescue workers has traditionally relied on linear routing plans, manually-reported position updates, and line-of-sight observation. These methods are labor-intensive, inaccurate, and insufficient for maintaining continuity of situation awareness for time-critical operations spanning substantial geographic areas.

To enhance personnel safety and improve operational coordination, a need exists for innovative technologies capable of automatically and continuously monitoring the physiological health metrics and precisely tracking the real-time locations of all active search and rescue workers. Comprehensive full-situation awareness of both environmental conditions and individual team member status represents a critical capability gap for modern search and rescue operations.

Recent advances in wearable sensors, wireless networking, and location-tracking technologies offer potential solutions for addressing this need. However, current implementations have seen limited effectiveness due to various technical hurdles, including body-worn sensor reliability, weather/terrain resilience, power management constraints, signal penetration issues, and the demands of secure data transmission from remote areas. Accordingly, there is a need for robust search and rescue personnel monitoring and tracking systems able to overcome these technical challenges to provide continuous health/location awareness for maximizing operational safety and efficacy.

### BRIEF DESCRIPTION OF THE DRAWINGS

The various advantages and features of the present technology will become apparent by reference to specific implementations illustrated in the appended drawings. A person of ordinary skill in the art will understand that these drawings show only some examples of the present technology and would not limit the scope of the present technology to these examples. Furthermore, the skilled artisan will appreciate the principles of the present technology as described and explained with additional specificity and detail through the use of the accompanying drawings in which:

FIG. 1 illustrates an exemplary garment having a pocket, according to some aspects of the disclosure.

FIG. 2 illustrates another exemplary garment having a pocket, according to some aspects of the disclosure.

FIG. 3 illustrates two exemplary garments having a pocket at different locations, according to some aspects of the disclosure.

FIG. 4 illustrates an exemplary sensor pocket, according to some aspects of the disclosure.

FIG. 5 illustrates another exemplary sensor pocket, according to some aspects of the disclosure.

FIG. 6 illustrates a view of an exemplary sensor pocket and exemplary electronics according to some aspects of the disclosure.

FIG. 7 illustrates a cross-sectional view of an exemplary sensor pocket and exemplary electronics according to some aspects of the disclosure.

FIG. 8 is a circuit diagram of an exemplary electronics system, according to some aspects of the disclosure.

FIG. 9A is a block diagram illustrating components of a remote monitoring system, according to some aspects of the disclosure.

FIG. 9B is a block diagram illustrating a method performed by a remote monitoring system, according to some aspects of the disclosure.

FIG. 10 is a block diagram illustrating operations performed by a remote monitoring system, according to some aspects of the disclosure.

FIG. 11 is a block diagram of an example computing device, according to some aspects of the disclosure.

FIGS. 12-13 depict exemplary screenshots of a frontend of the remote monitoring system, according to some aspects of the disclosure.

FIGS. 14-18 are photographs of an exemplary garment having an exemplary sensor pocket, according to some aspects of the disclosure.

FIG. 19 is a photograph of an exemplary electronics system, according to some aspects of the disclosure.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a more thorough understanding of the subject technology. However, it will be clear and apparent that the subject technology is not limited to the specific details set forth herein and may be practiced without these details. In some instances, structures and components are shown in block diagram form to avoid obscuring the concepts of the subject technology.

### Overview of Problem

Emergency services operations such as search and rescue often take place in physically demanding locations and situations. In conversations with search and rescue personnel, one recurrent issue was the need to be able to monitor the physical well-being and vital signs of search and rescue personnel while they are out on a mission. Search and rescue personnel themselves should not require rescue.

Search and rescue missions now often use Geographic Information Systems (GIS) mapping applications such as SARTopo (from the company CALTopo) to track the location via Global Positioning System (GPS) of SAR personnel while they are on the field. This tracking occurs from the command post.

However, search and rescue personnel noted that there were no devices available that met the criteria for being able to both track the location of personnel while also monitoring their well-being while they were out on the field. While there are various devices that do some parts of this such as a fitness smart watch or smartphone, there is no system that is actually capable of meeting the needs of search and rescue personnel.

One reason is that such devices and systems need to be simple to use and not complicate the mission. For example, feedback from search and rescue and emergency services personnel reinforced the importance of systems that were simple and that were easy to integrate into existing practices and require minimal attention from the user. For example, personnel gave examples of how firefighters pulled unconscious people from burning buildings even though they were wearing an oxygen monitor because they had forgotten or disregarded turning on the oxygen monitor.

SAR operations often take place in hazardous conditions. Through interviews with SAR personnel, a long-felt but unmet need exists for a system to monitor the well-being of SAR personnel while they are out on a mission locating people who are lost or injured in disasters.

### Summary of Overall Solution and Applications

The system addresses the problem by creating a base layer garment with integrated sensors that can wirelessly upload location and vital signs data to a GIS mapping website in such a way that at a command post, the location and well-being of personnel can be monitored in real-time. The garment with biometric sensors can monitor the vital signs of the user and publish those data onto a GPS software in such a way that the viewer of the software can see the geographic location and health and well-being of the person. The overall system helps SAR officers by making sure SAR personnel are safe. The system can help make sure lost people are found faster and the process will work more smoothly.

The present disclosure describes a garment that includes a removable electronic device that can sense body core temperature, heart rate, and geo-spatial location of the wearer, and transmit this data wirelessly to a database accessible to a GIS application, so that a viewer of the app can track the location and biometric data of the wearer. The location of the wearer is tracked in real-time as a line on a GIS map; the biometric data is mapped onto the geo-location data. The color of the line changes in response to biometric data.

This is a system that has both software and hardware components. It can be thought of as having three components. The first component is a base layer garment that contains the vital signs sensor system hardware. The second component is a hardware component that is carried by the garment which contains the sensors, microprocessor and transmitters. The third aspect is a software remote monitoring system that enables the location and vital signs information to be transmitted onto a mapping application so that the location and well-being of the wearer can be remotely monitored.

Although the system was developed in response to an unmet need in the SAR/emergency services community, the system has applications in other areas where remote sensing and data logging of vital signs in potentially difficult environments is of value, such as monitoring the whereabouts and well-being of people living with conditions such as Alzheimer's.

The term “garment” herein refers to any wearable form of the device, and not solely the specific embodiments described. Examples of garments can include, but are not limited to, shirts, blouses, t-shirts, tank tops, tunics, dresses, skirts, trousers, jeans, leggings, shorts, capris, suits, jackets, coats, vests, sweaters, cardigans, hoodies, sweatshirts, underwear, bras, camisoles, slips, boxer shorts, briefs, panties, socks, stockings, tights, swimsuits, bikinis, trunks, singlets, robes, pajamas, nightgowns, raincoats, ponchos, overalls, rompers, jumpsuits, scarves, shawls, ties, bow ties, gloves, mittens, hats, caps, beanies, turbans, veils, headbands, belts, boots, shoes, sandals, clogs, sneakers, moccasins, loafers, slippers, athletic gear, jerseys, compression wear, uniforms, protective clothing, aprons, lab coats, coveralls, and flame-resistant suits.

### Overview: Garment Design and Electronics That can be Integrated With the Garment

The present disclosure describes a system for remote real-time tracking of a wearer's vital signs and location. A garment can include sensors to detect vital signs and location of the wearer, enabling the data to be wirelessly transmitted to the Internet in such a way that the data can be viewed in real-time on a GIS mapping application. A garment can include a sensor module with sensors for vital signs and location. The electronics can wirelessly communicate with a smartphone and/or a wireless network.

A one piece device having sensors, other circuitry, and a battery can be encased in a water-resistant envelope that can be easily inserted and removed from a garment. The above further includes a photosensitive blood pulse and oxygen sensor. The pocket and envelope may include an optical window that is sufficiently transparent to enable the detection of pulse and blood oxygen when held against the skin of the wearer. The system may be configured such that when inserted into special pockets in a garment, enables the sensors to be located in suitable positions to sense accurate data.

The system includes a battery powered electronic device with a built-in battery and inductive wireless charging capabilities so that the device can be contained within a waterproof envelope. In other words, the battery does not need to be taken out in order to be charged. The battery can be charged even when it is contained in the waterproof envelope. The electronics are powered by a 5 v lithium-ion battery that can be charged wirelessly. Integrated wireless charging means that the system can be ready at all times and does not need to be removed to be charged. The electronics do not need to be taken out of the pocket to be charged.

The system is self-activated when the garment is donned. The device can be automatically activated when the garment is pulled on and begins sensing vital signs data. For example, the electronics system can be installed in a base layer garment that remains on in a dormant state that is activated when the thermistors detect ranges in the body temperature and the pulse meter detects signal resembling a pulse. A device that becomes activated when the wearer pulls the garment on, enabling the sensors to begin monitoring vital signs and transmitting location. This is important for emergency services operations where it is beneficial to have a minimum number of readiness actions required.

A garment with pockets and sleeves made of an elastic material that can hold an electronic sensing device against the body of the wearer maintaining consistent contact with the skin. In one example, a photo optical sensor can be located on the inner arm/bicep. In one example, a photo optical sensor can be located on the upper chest. The core temp sensing thermistor is positioned under the arm. The photo optical sensor can be co-located with the transmitter, receiver and battery charging system. The core temp sensing thermistor is located on a single lead, enabling the electronic device to be optimally positioned. An electronic device as described where the photo optical sensor, microprocessor, transceiver, and battery charging mechanism are all co-located with an attached thermistor on a lead of sufficient length to enable both the thermocouple and integrated board to be optimally located.

The device as described above where all the components including the lead can be contained in a waterproof/water-resistant envelope. The water-resistant envelope can be made of a range of materials such a transparent thermoplastic film, sealed together with stitching and/or heat adhesive. A water-resistant sleeve that can be opened to service electronic devices such as replacing battery and/or repair. The sensors, microprocessor, battery and charging are integrated into a flexible water-resistant housing, which can be easily removed as a unit for service or garment washing. A garment with pockets configured so that the sensing device can be easily inserted or removed for cleaning or servicing. The device is packaged so that it can be easily slipped into or removed from the garment for servicing or for the garment to be cleaned.

A garment can include sleeves or pockets configured in multiple ways to facilitate the installation and removal of an electronic device. This can include elastic openings with zipper closure, Velcro, and other similar fasteners. A sleeve or pocket with a transparent window or opening to enable a photo optical device to illuminate and view the skin of the wearer in such a way that pulse and blood oxygen level of the wearer can be monitored. The system is incorporated into the garment in ways that are low profile and do not impede essential physical activity.

### Overview: Remote Monitoring System

The present disclosure describes various aspects of a system that can remotely track in real-time the location and well-being status of SAR personnel while they are on a mission. The system wirelessly uploads this data to mapping apps used by SAR teams, such as SARTopo, so that viewers at a command post can easily monitor both the location and well-being of to get information of vital signs on SAR personnel while they are out on a mission. The system is simple to use and maintain and graphically presents the data in easy to interpret form.

An application programming interface (API) can be implemented to receive the data transmitted wirelessly by an electronic device. Via the API, data collected by the electronics system can be utilized by computer applications so that the data can be remotely viewed.

The remote monitoring platform can remotely track the location and well-being of an individual in real-time. The electronics system may implement an algorithm that converts data ranges from sensors into color values registered in real-time on a GIS mapping application. The algorithm can change a status color displayed to a user in response to the health status of the person being tracked changing. For example, if the vital sign sensor values are within what are set as safe range, the GPS track icon appears as green. If the sensor values are what are predetermined to be at risk, the GPS track icon appears as yellow. If the vital signs data are within what are a predetermined danger threshold, the GPS track icon appears red. The system includes a unique algorithm that interprets the vital signs data and converts the GPS track icon into colors of green, yellow, or red so that the viewer can easily monitor the well-being in real-time.

The system can include one or more of: a base layer garment incorporating multiple sensors that collect core temperature, heart rate, hydration, SpO2 (peripheral oxygen saturation measured using a pulse oximeter), and GPS location, and a microprocessor to convey the information either directly or through SAR personnels' phones, which push the vitals data into SARTopo, a SAR mapping software. The system may transmit data from a garment to a website either through a cell phone or directly to the cellular network.

The system includes one or more pathways for transmitting the data from the device to a website where it can be accessed by a suitable GIS mapping application. The system in its simplest form can use a cell phone as a relay between the device and the application. However, because cell networks are not always reliable, especially in remote locations, the system can communicate GPS and vital signs data wirelessly without requiring a smartphone. In some embodiments, the device transmits the data to backend system by connecting to a smart phone through Bluetooth, and the smart phone forwards the data to the backend system. In some embodiments, the device transmits the data to the backend system wirelessly without going through a cellular connection. In some embodiments, the device transmits the data directly to the backend system through a cellular connection.

### Garment Design With Pocket(s) and Electronics to be Embedded in the Pocket

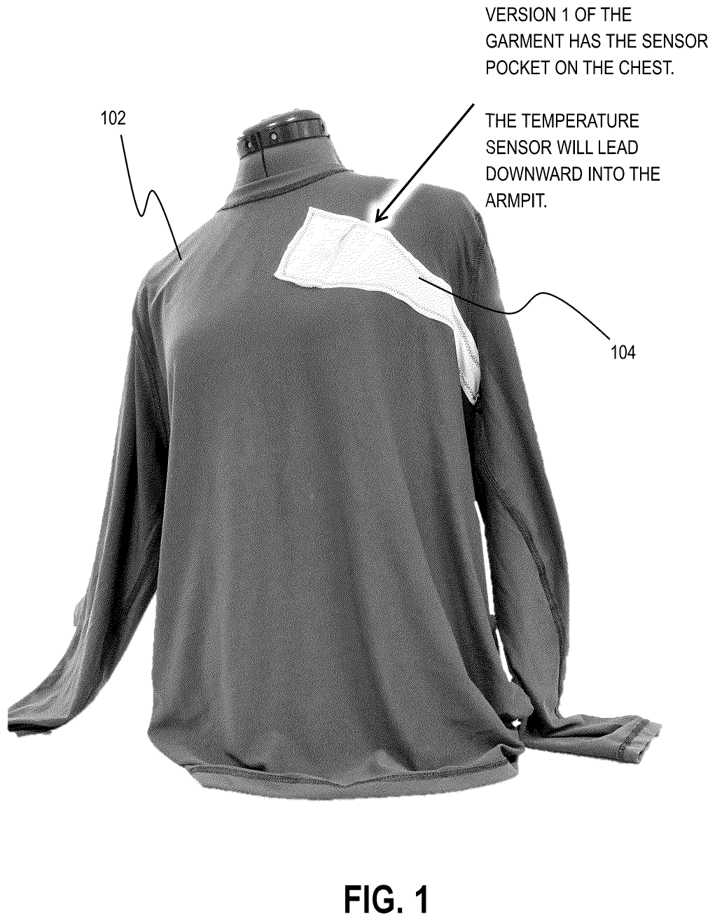

FIG. 1 illustrates exemplary garment 102 having pocket 104, according to some aspects of the disclosure. Garment 102 may be a base layer. As shown, garment 102 is turned inside out. Pocket 104 is located in the front on the chest area. Temperature sensor (e.g., thermistor) may lead downward into the armpit.

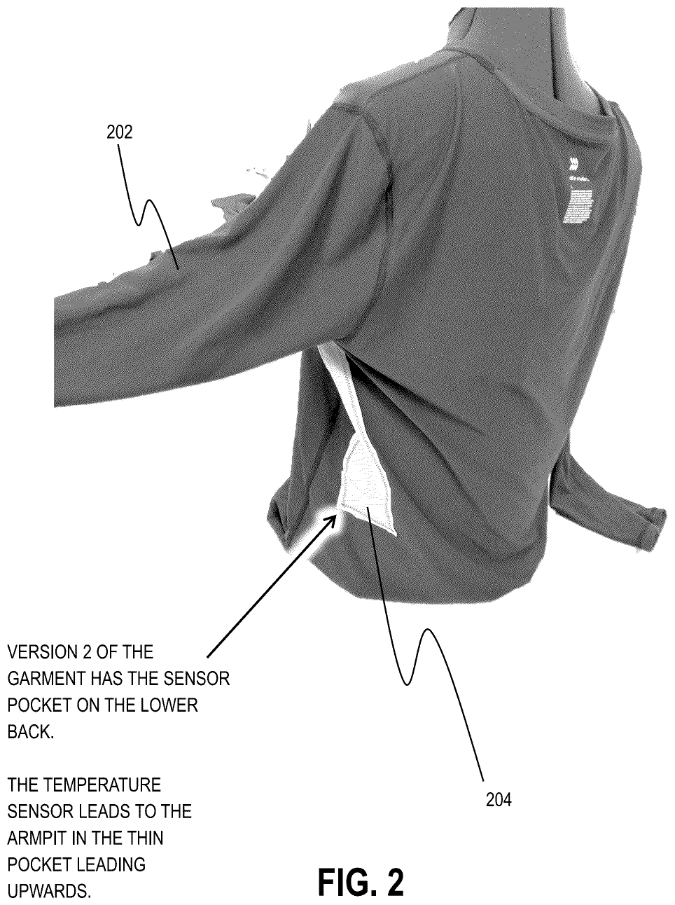

FIG. 2 illustrates exemplary garment 202 having pocket 204, according to some aspects of the disclosure. Garment 202 may be a base layer. As shown, garment 202 is turned inside out. Pocket 204 is located on the side in the lower back area. Temperature sensor (e.g., thermistor) may lead upward into the armpit.

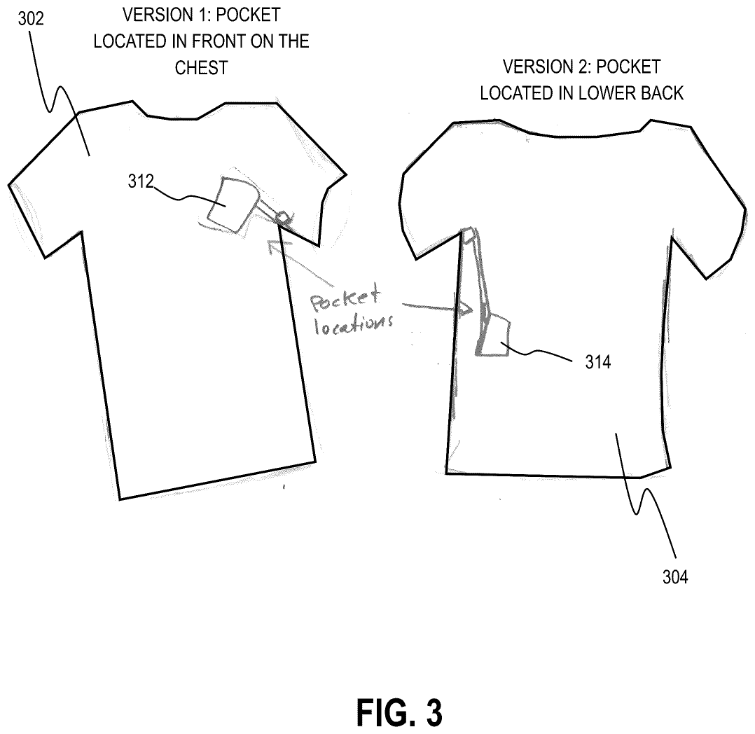

FIG. 3 illustrates two exemplary garments having a pocket at different locations, according to some aspects of the disclosure. Garment 302 on the left side illustrates pocket 312 located in the front on the chest area. Garment 304 on the right side illustrates pocket 314 located in the lower back area.

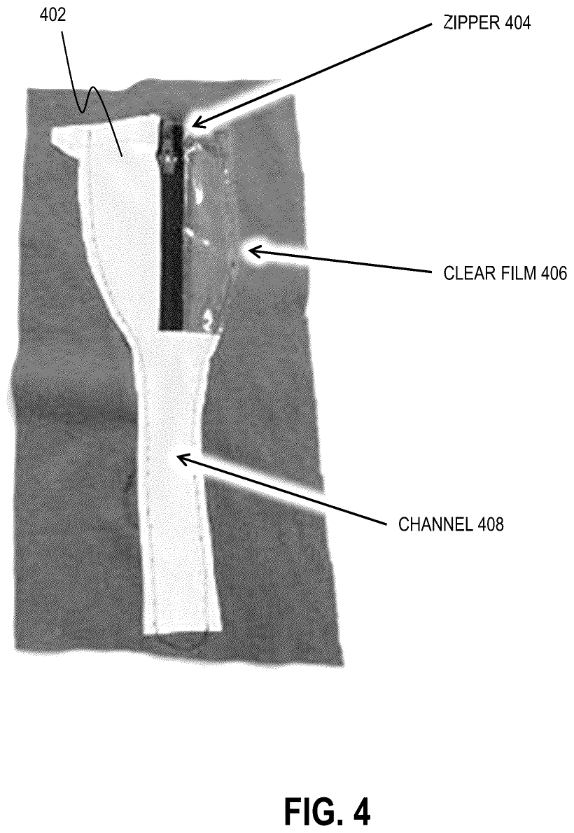

FIG. 4 illustrates exemplary sensor pocket 402, according to some aspects of the disclosure. Sensor pocket 402 may be sewn or affixed to the garment on the side (e.g., on the inside) that faces the skin of the wearer of the garment. Sensor pocket 402 may include zipper 404. Zipper 404 may allow for easy removal and insertion of the electronics. Sensor pocket 402 may include clear film 406 to let the optical sensor see through to the skin. Sensor pocket 402 may include channel 408. Channel 408 may lead to the armpit for the thermistor. The fabric of the sensor pocket may be stretch/elastic fabric.

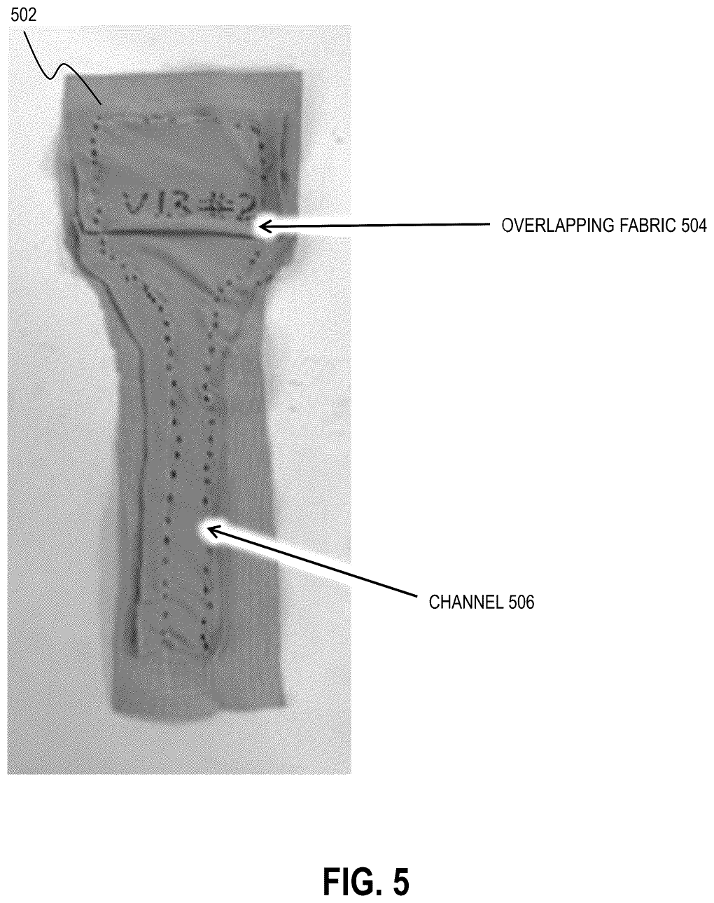

FIG. 5 illustrates another exemplary sensor pocket 502, according to some aspects of the disclosure. Sensor pocket 502 may be sewn or affixed to the garment on the side (e.g., on the inside) that faces the skin of the wearer of the garment. Sensor pocket 502 may include overlapping fabric 504 to keep the electronics in place or secured within the pocket. Sensor pocket 502 may include channel 506. The channel may lead to the armpit for the thermistor to sense temperature. The fabric of the sensor pocket may be stretch/elastic fabric.

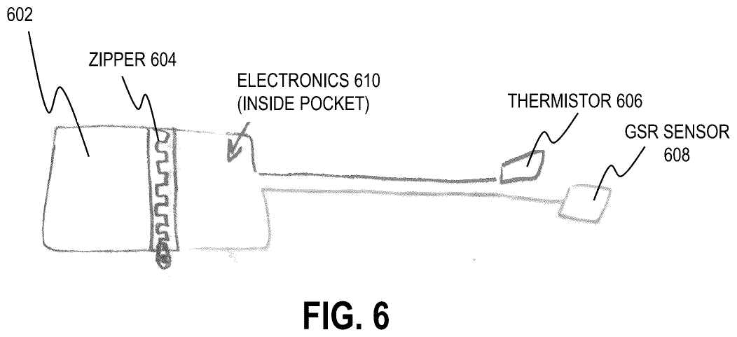

FIG. 6 illustrates a view of exemplary sensor pocket 602 and exemplary electronics 610 according to some aspects of the disclosure. Electronics 610 (on a printed circuit board) may be in a first part of sensor pocket 602 on which zipper 604 may be provided. Thermistor 606 and galvanic skin response (GSR) sensor 608 may be connected to the printed circuit board via wires. Thermistor 606 and GSR sensor 608 may be embedded in a channel of sensor pocket 602 (channel is not depicted). The printed circuit board for electronics 610, wires, and sensors (e.g., thermistor 606 and GSR sensor 608) can be taken out of pocket 602 for easy access, and can make the garment washable.

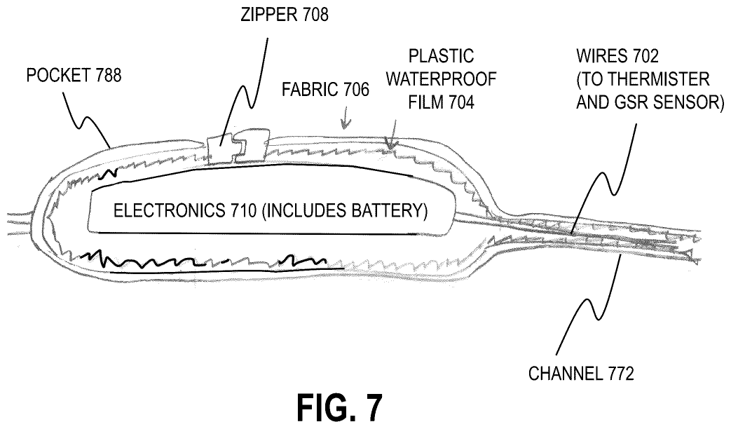

FIG. 7 illustrates a cross-sectional view of exemplary sensor pocket 788 and exemplary electronics 710 according to some aspects of the disclosure. Electronics 710 (with printed circuit board) may be in a first part of sensor pocket 788 on which zipper 708 may be provided. The electronics may include a battery. The thermistor and GSR sensor may be connected to the printed circuit board of the electronics 710 via wires 702. The thermistor and GSR sensor may be embedded in channel 772 of sensor pocket 788. Electronics 710 (with the battery and printed circuit board) and wires 702 to thermistor and GSR sensors may be wrapped by plastic waterproof film 704.

A base layer garment is preferably made of a wicking fabric. The base layer may fit snuggly enough so that the sensors can be held in position against the body. The sensor device, which can include the electronics, the printed circuit board, the wires to sensors, and sensors, can be at least partially wrapped in plastic waterproof film. The sensor device is slipped into pockets in the garment that optimally position the sensors. There are a number of configurations for these pockets/sleeves that enable the sensor device to be easily slipped in and out of the garment. The pockets/sleeves have provisions to enable the optical based sensors an optical view of the skin surface either through an opening or the use of a transparent material.

There are a number of locations for these pockets/sleeves. Locations include for example a sleeve shaped so that when being worn, a temperature sensor (thermistor) is positioned in the armpit and the optical heart rate sensor is facing the skin and has an optical pathway to the skin surface. The bulkiest parts of the sensor system are located in areas that provide minimum obstruction to the activity of the user during operations. These might include, but are not limited to the inside of the upper arm, on the lower back. The garment is designed so that it can be easily put on and easy to prepare for operation. For example, the garment is always on, but the system is in a sleep mode. The garment turns on when the sensors detect body temperature values or data representing a pulse. The system turns on when you put the garment on.

In some embodiments, a sensor pocket may have dimensions of 9 cm wide by 28 cm long.

In some embodiments, the sensor pocket includes a window open for the optical sensor to see through.

In some embodiments, the sensor pocket can be located on the upper chest.

In some embodiments, the sensor pocket can be located on the lower back.

In some embodiments, the sensor pocket can be located on the bicep.

In some embodiments, the fabric for the pocket is made for a tight but comfortable fit, and a lot of stretchability. The pocket is a sewn-in pocket on the inside of a base layer. The pockets can be made using stretchable fabrics and zippers. The electronics system is waterproof because of the heat sealing plastic that is sewn around the sensors. There are also “windows” in the fabric that allow the sensors to directly view the skin for better measurements. The circuit board itself is packaged in a way so that it does not move around or get displaced when the wearer moves.

### Circuitry and Electronics System

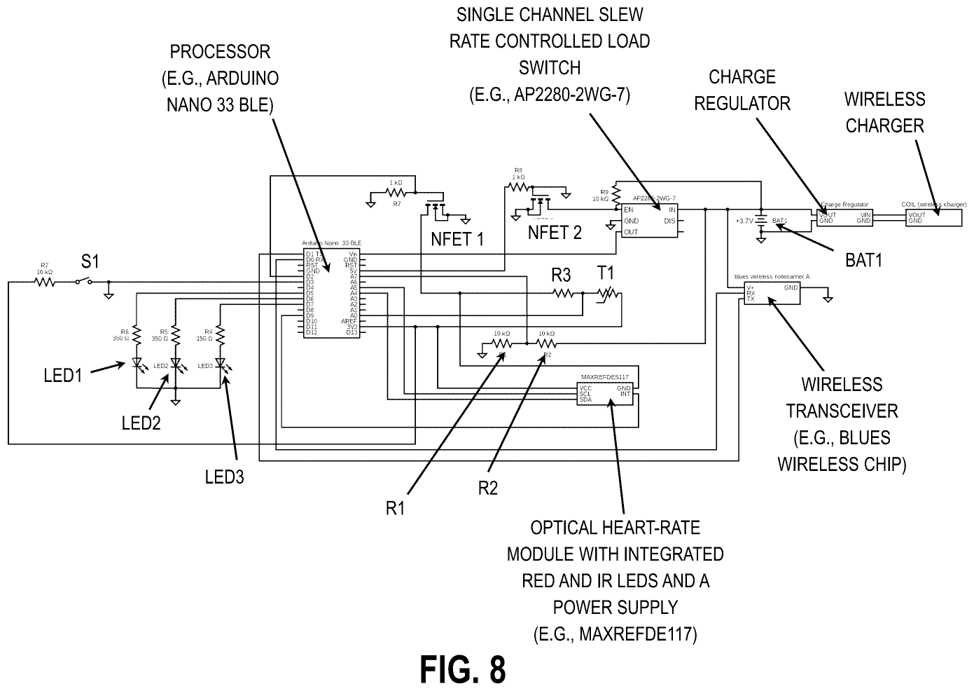

FIG. 8 is a circuit diagram of an exemplary electronics system, according to some aspects of the disclosure.

The symbol labeled “NFET1” switches a ground connection to the two main sensors (thermistor and MAXREFDE117). This means they will not consume power during low power mode as the connection to ground will be floating.

The symbol labeled “NFET2” switches connection to ground to pull down the input to the “enable” pin on the AP2280-2WG-7. This blocks power from flowing to the battery when the processor (e.g., Arduino Nano 33 BLE microcontroller) has a Universal Serial Bus (USB) connection. By default, the “enable” pin is high meaning it allows power through until the USB is plugged in.

On the far left there is a symbol labeled “S1”. It is the switch used to toggle low power mode.

Next to the switch there are three symbols labeled “LED1”-“LED3”. Those are the LEDs used to indicate power levels.

The symbols labeled “R1” and “R2” are used in a voltage divider to monitor the voltage of the battery.

The symbols labeled “R3” and “T1” are used in the voltage divider circuit to measure the temperature. “T1” is the thermistor. The thermistor for sensing core temperature is attached to the board by a lead long enough to position the sensor within the armpit.

The battery is the symbol labeled “BAT1”. The battery may be rechargeable via an inductive charging coil.

In some embodiments, the circuitry further includes a hydration level sensor. Hydration level sensor can sense skin surface moisture level. In one example, the hydration level sensor includes one or more electrodes to measure skin conductivity and/or resistance of a fabric pad that can absorb sweat or moisture from the skin.

In some embodiments, the circuitry further includes an accelerometer.

In some embodiments, the circuitry further includes a gyroscope.

In some embodiments, the circuitry further includes a thermistor.

In some embodiments, the circuitry further includes a barometric pressure sensor.

### Remote Monitoring Platform

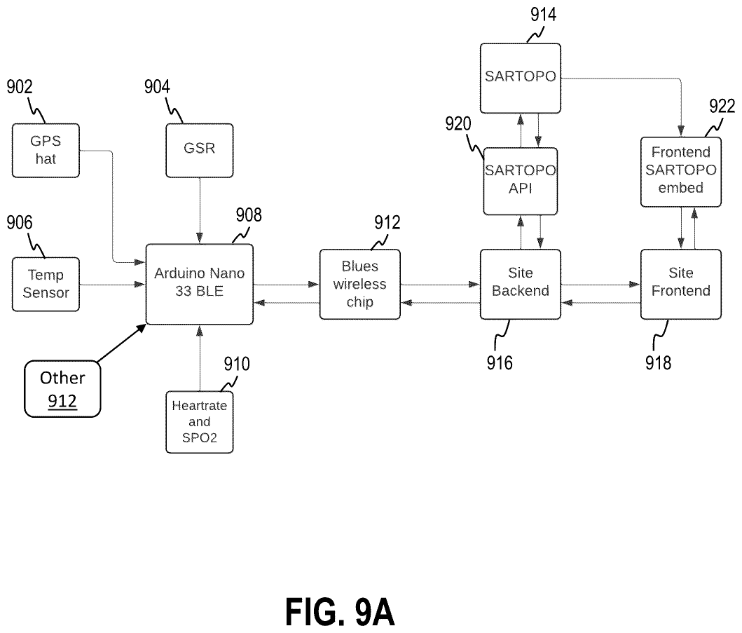

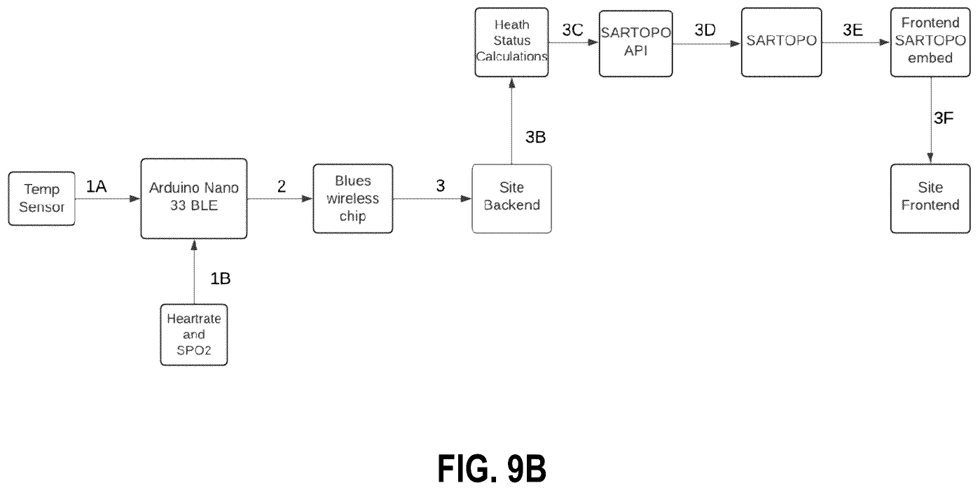

FIG. 9A is a block diagram illustrating components of a remote monitoring system, according to some aspects of the disclosure.

Arduino Nano 33 BLE: “Motherboard” having a processor that connects all sensors together to transfer data.

GPS hat 902 is a GPS attachment to the processor.

GSR 904 is a skin moisture sensor. It is run by the processor and sends its data to the Blues Wireless Chip. GSR sensor can measure skin moisture/hydration levels. In one example, the hydration level sensor includes one or more electrodes to measure conductivity and/or resistance of a fabric pad that can absorb sweat or moisture from the skin.

Temp Sensor 906 is a thermistor. It is run by the processor and sends its thermal data to the Blues Wireless Chip.

Other 912 includes other sensor(s) can include one or more of: an accelerometer, a gyroscope, a barometric pressure sensor.

Heart Rate and SpO2 910 can be a MAXREFDES117 sensor (optical heart rate, SpO2, temperature sensor). It is run by the processor and sends the user's heart rate and SPO2 information to the Blues Wireless Chip.

Blues wireless chip 912 is Blues wireless note carrier A 2.0. It is run by the processor. It sends data to the website and gets GPS data. A chipset with integrated sim card and GPS to send data to compatible devices.

Site backend 916 processes data sent by the Blues note carrier. It relays data to the SARTopo map via an application programming interface (API). It runs on flask and python. A database that collects information being transmitted from the Blues board. The backend code of site backend 916 can include health status calculations. It takes the sent health data from the Blues Wireless Chip and converts it to a relative health status. It is sent to SARTopo 914 via SARTopo API 920.

SARTopo API 920 is a python API that can interact with SARTopo maps. It is used to update the map in real-time with health data.

SARTopo 914 is a map hosting software that is currently used in many search and rescue situations. Real-time data is incorporated into the SARTopo map that local search and rescuers are using currently.

Site Frontend 918 displays data in a user friendly way via a table underneath the map. Also, hosts the SARTopo map shown via an HTML embed.

Frontend SARTopo Embed 922 is an embed of the SARTopo map which is displayed on the site frontend 918 and is updated live by the health data.

FIG. 9B is a block diagram illustrating a method performed by a remote monitoring system, according to some aspects of the disclosure.
1A: data from the temperature sensor (thermistor) goes to the processor to be processed and sent.
1B: data from the heart rate and SpO2 (MAXREFED117) sensor to the processor to be processed and sent.

Other data from other sensor(s) can be transmitted to the processor in the same manner as 1A and 1B.
2: all data (heart rate, temperature, SpO2, location, etc.) is given to the Blues wireless notecard to be sent.
3: Blues wireless notecard sends the data from the processor to the backend of the site
3B: part of the backend program takes the health data and processes it into one general health status (good, neutral, bad).
3C: sends location change requests and color change requests to the SARTopo API to update the location tracker.
3D: SARTopo API sends to the SARTopo map.
3E: SARTopo map sends data to the embed in the frontend code.
3F: SARTopo map data is displayed via the embed.

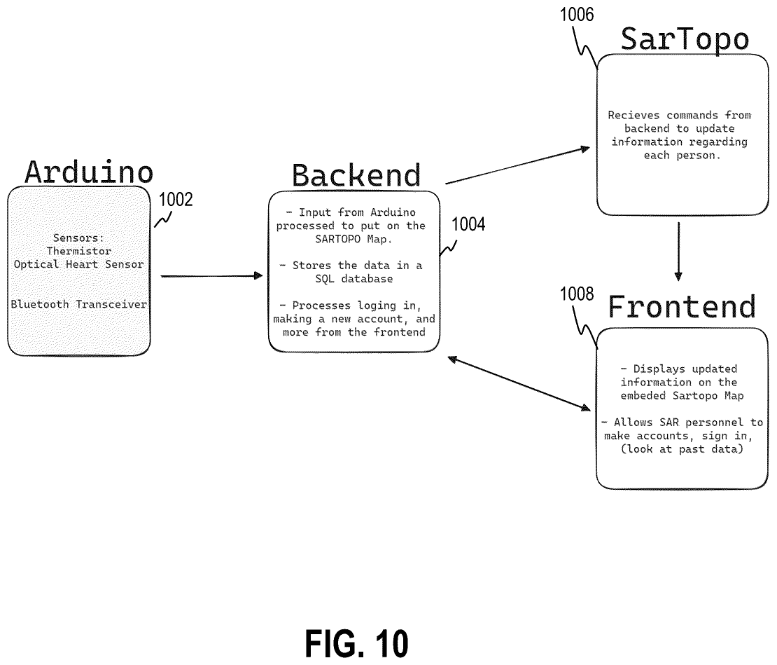

FIG. 10 is a block diagram illustrating operations performed by a remote monitoring system, according to some aspects of the disclosure.

Backend 1004 receives input from processor 1002. The input may be processed to put on the SARTopo map 1006.

Backend 1004 stores the data in a database.

Backend 1004 processes logging in, making a new account, and more from the frontend 1008.

SARTopo map 1006 receives commands from backend 1004 to update information regarding each person.

Frontend 1008 displays updated information on the embedded SARTopo map.

Frontend 1008 allows SAR personnel to make accounts, sign in, and look at past data.

### Exemplary Computing Device

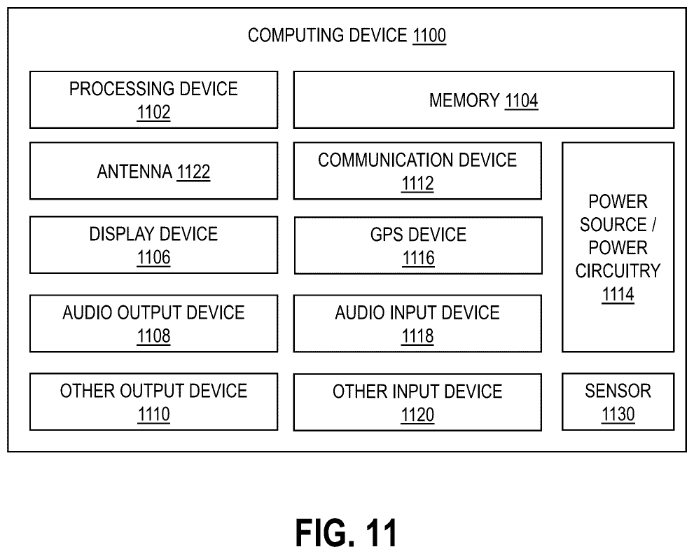

FIG. 11 is a block diagram of an example computing device 1100, in accordance with various embodiments. In some embodiments, the computing device 1100 can be used as at least part of the circuitry and electronics system illustrated in FIG. 8. In some embodiments, the computing device 1100 can be used as at least part of the remote monitoring system illustrated in FIGS. 9-10. A number of components are illustrated in FIG. 11 as included in computing device 1100, but any one or more of these components may be omitted or duplicated, as suitable for the application. In some embodiments, some or all of the components included in the computing device 1100 may be attached to one or more motherboards. In some embodiments, some or all of these components are fabricated onto a single system on a chip (SoC) die. Additionally, in various embodiments, the computing device 1100 may not include one or more of the components illustrated in FIG. 11, but the computing device 1100 may include interface circuitry for coupling to the one or more components. For example, the computing device 1100 may not include a display device 1106, but may include display device interface circuitry (e.g., a connector and driver circuitry) to which a display device 1106 may be coupled. In another set of examples, the computing device 1100 may not include an audio input device 1118 or an audio output device 1108 but may include audio input or output device interface circuitry (e.g., connectors and supporting circuitry) to which an audio input device 1118 or audio output device 1108 may be coupled.

The computing device 1100 may include a processing device 1102 (e.g., one or more processing devices). The processing device 1102 processes electronic data from registers and/or memory to transform that electronic data into other electronic data that may be stored in registers and/or memory. The computing device 1100 may include a memory 1104, which may itself include one or more memory devices such as volatile memory (e.g., DRAM), nonvolatile memory (e.g., read-only memory (ROM)), high bandwidth memory (HBM), flash memory, solid state memory, and/or a hard drive. In some embodiments, the memory 1104 may include memory that shares a die with the processing device 1102. In some embodiments, the memory 1104 includes one or more non-transitory computer-readable media storing instructions executable to perform operations for method illustrated in FIG. 10, or some operations performed by one or more components of the circuitry and electronics system illustrated in FIG. 8, or some operations performed by one or more components of the remote monitoring system illustrated in FIGS. 9-10, or some operations described herein. The instructions stored in the one or more non-transitory computer-readable media may be executed by the processing device 1102.

In some embodiments, the computing device 1100 may include a communication chip 1112 (e.g., one or more communication chips). For example, the communication chip 1112 may be configured for managing wireless communications for the transfer of data to and from the computing device 1100. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not.

The communication chip 1112 may implement any of a number of wireless standards or protocols, including but not limited to Institute for Electrical and Electronic Engineers (IEEE) standards including Wi-Fi (IEEE 802.10 family), IEEE 802.16 standards (e.g., IEEE 802.16-2005 Amendment), Long-Term Evolution (LTE) project along with any amendments, updates, and/or revisions (e.g., advanced LTE project, ultramobile broadband (UMB) project (also referred to as “3GPP2”), etc.). IEEE 802.16 compatible Broadband Wireless Access (BWA) networks are generally referred to as WiMAX networks, an acronym that stands for worldwide interoperability for microwave access, which is a certification mark for products that pass conformity and interoperability tests for the IEEE 802.16 standards. The communication chip 1112 may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication chip 1112 may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chip 1112 may operate in accordance with Code-division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. The communication chip 1112 may operate in accordance with other wireless protocols in other embodiments. The computing device 1100 may include an antenna 1122 to facilitate wireless communications and/or to receive other wireless communications (such as AM or FM radio transmissions).

In some embodiments, the communication chip 1112 may manage wired communications, such as electrical, optical, or any other suitable communication protocols (e.g., the Ethernet). As noted above, the communication chip 1112 may include multiple communication chips. For instance, a first communication chip 1112 may be dedicated to shorter-range wireless communications such as Wi-Fi or Bluetooth, and a second communication chip 1112 may be dedicated to longer-range wireless communications such as GPS, EDGE, GPRS, CDMA, WiMAX, LTE, EV-DO, or others. In some embodiments, a first communication chip 1112 may be dedicated to wireless communications, and a second communication chip 1112 may be dedicated to wired communications.

The computing device 1100 may include battery/power circuitry 1114. The battery/power circuitry 1114 may include one or more energy storage devices (e.g., batteries or capacitors) and/or circuitry for coupling components of the computing device 1100 to an energy source separate from the computing device 1100 (e.g., AC line power).

The computing device 1100 may include a display device 1106 (or corresponding interface circuitry, as discussed above). The display device 1106 may include any visual indicators, such as a heads-up display, a computer monitor, a projector, a touchscreen display, a liquid crystal display (LCD), a light-emitting diode display, or a flat panel display, for example.

The computing device 1100 may include an audio output device 1108 (or corresponding interface circuitry, as discussed above). The audio output device 1108 may include any device that generates an audible indicator, such as speakers, headsets, or earbuds, for example.

The computing device 1100 may include an audio input device 1118 (or corresponding interface circuitry, as discussed above). The audio input device 1118 may include any device that generates a signal representative of a sound, such as microphones, microphone arrays, or digital instruments (e.g., instruments having a musical instrument digital interface (MIDI) output).

The computing device 1100 may include a GPS device 1116 (or corresponding interface circuitry, as discussed above). The GPS device 1116 may be in communication with a satellite-based system and may receive a location of the computing device 1100, as known in the art.

The computing device 1100 may include another output device 1110 (or corresponding interface circuitry, as discussed above). Examples of the other output device 1110 may include an audio codec, a video codec, a printer, a wired or wireless transmitter for providing information to other devices, or an additional storage device.

The computing device 1100 may include another input device 1120 (or corresponding interface circuitry, as discussed above). Examples of the other input device 1120 may include an accelerometer, a gyroscope, a compass, an image capture device, a keyboard, a cursor control device such as a mouse, a stylus, a touchpad, a bar code reader, a Quick Response (QR) code reader, any sensor, or a radio frequency identification (RFID) reader.

The computing device 1100 may have any desired form factor, such as a handheld or mobile computer system (e.g., a cell phone, a smart phone, a mobile Internet device, a music player, a tablet computer, a laptop computer, a netbook computer, an ultrabook computer, a personal digital assistant (PDA), an ultramobile personal computer, etc.), a desktop computer system, a server or other networked computing component, a printer, a scanner, a monitor, a set-top box, an entertainment control unit, a vehicle control unit, a digital camera, a digital video recorder, or a wearable computer system. In some embodiments, the computing device 1100 may be any other electronic device that processes data.

### Screenshots of Site Frontend

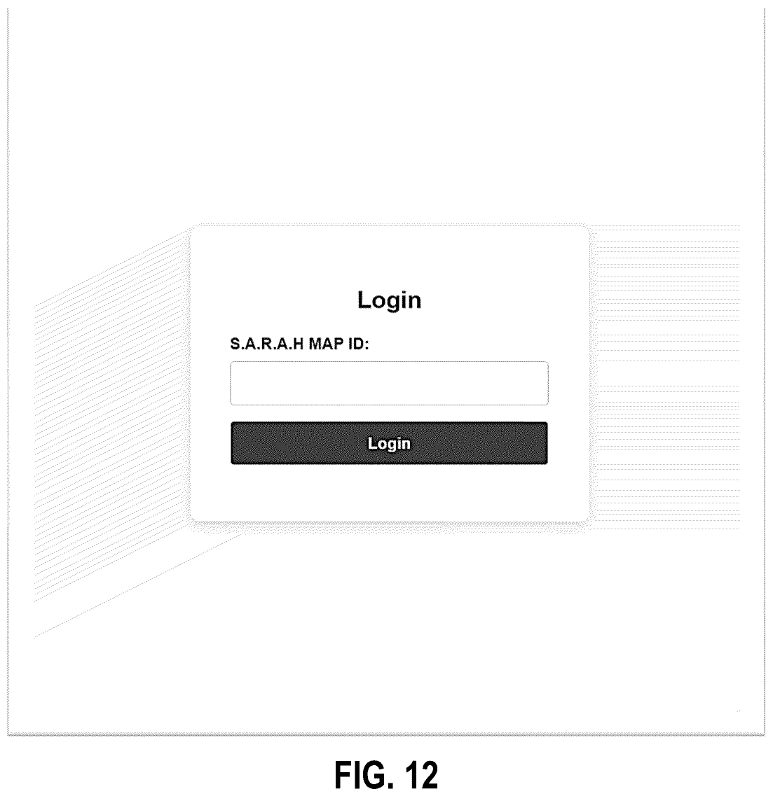

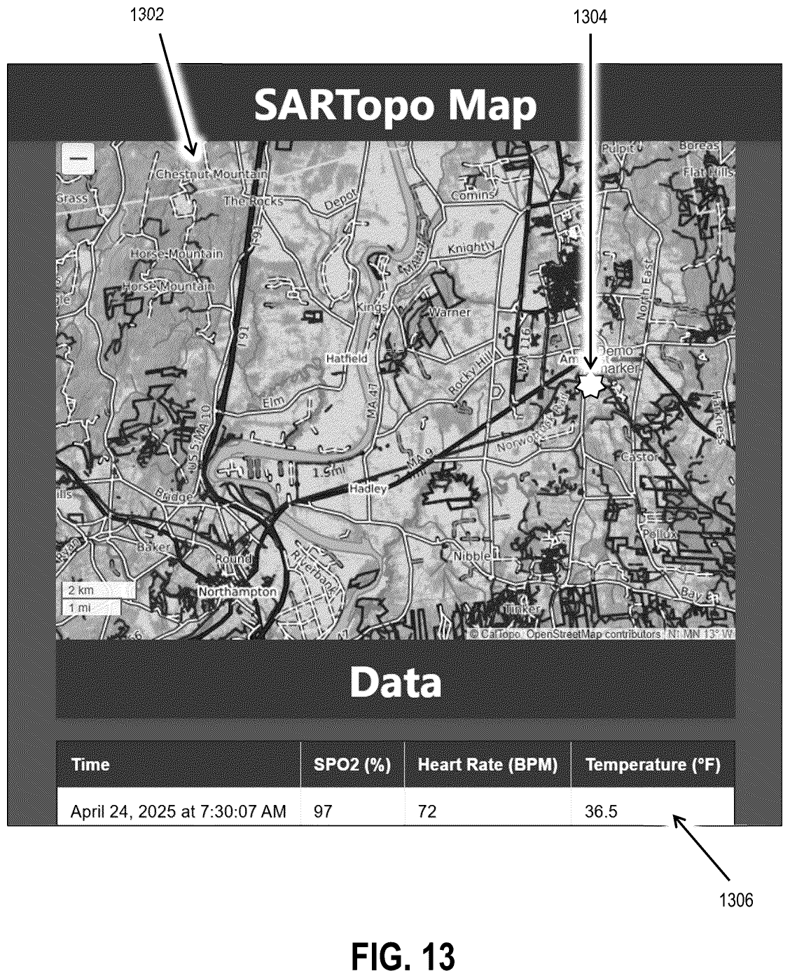

FIGS. 12-13 depict exemplary screenshots of a frontend of the remote monitoring system (e.g., Site Frontend 918 of FIG. 9A), according to some aspects of the disclosure. FIG. 12 is a screen allowing a user to access a particular map using a map identifier (“S.A.R.A.H MAP ID”). FIG. 13 is a screen displaying an embedded SARTopo map 1302, a GPS track icon 1304, and data 1306 about a person. GPS track icon 1304 can have different colors to indicate health status. Data 1306 can include vital signs data and/or a summary about the health status of the person.

### Variations on Garment Design With Pocket(s)

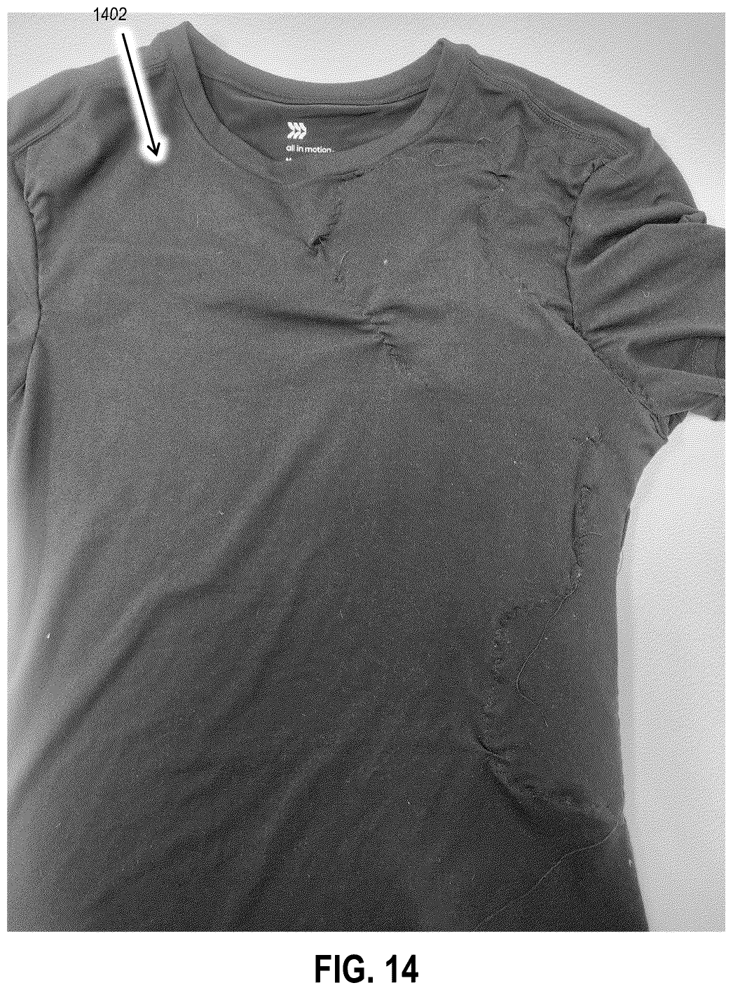

FIGS. 14-18 are photographs of exemplary garment 1402 having an exemplary sensor pocket, according to some aspects of the disclosure.

FIG. 14 is a photograph of a portion of garment 1402. Exemplary garment 1402 may be a base layer. As shown, garment 1402 is right side out. Pockets are not visible and are provided on the inside of exemplary garment 1402. Exemplary garment 1402 is made of a stretchy, athletic material to maintain comfort, range of motion, and to hold sensors properly in place during physical activity.

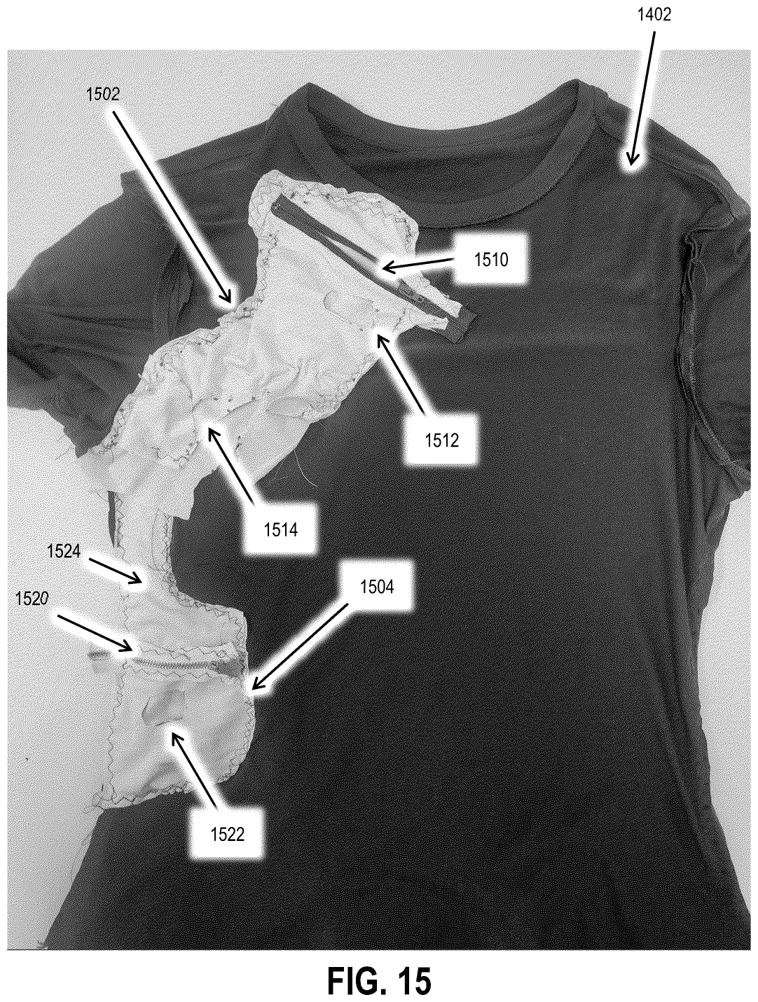

FIG. 15 is a photograph of a portion of garment 1402. As depicted, exemplary garment 1402 is turned inside out to reveal two pockets, e.g., pocket 1502, and pocket 1504. There are two separate pockets on the front left side of the garment in order to accommodate for an individual's personal comfort preferences as well as any other undergarments such as sports bras that may block a sensor's access to the skin and impair the garment's accuracy. Pocket 1502 and pocket 1504 on garment 1402 support the user's personal preference and work around any other undergarments that may be present. Pocket 1502 is located on the front left chest, and pocket 1504 is located on the front right midsection. Both pocket 1502 and pocket 1504 have a main section or portion to carry and secure most of the hardware and sensors, as well as a gradually narrowing pocket, referred to herein as channel 1514 and channel 1524, leading to the left armpit. Channel 1514 and channel 1524 can ensure connecting wire(s) do not bunch up and the sensor(s) at the end of the wire stays in place on the user's body.

Pocket 1502 is located in the front left on the chest area. Temperature sensor (e.g., thermistor) may lead downward into the armpit. Pocket 1502 can include zipper 1510, and opening 1512. Pocket 1502 can have channel 1514 for enclosing a wire connecting a thermistor to the electronics and/or a wire connecting a GSR sensor to the electronics. Channel 1514 can ensure that the sensors are positioned at an optimal location on the body for sensing.

Pocket 1504 is located in at the waist area on the left side. Temperature sensor (e.g., thermistor) may lead upward into the armpit. Pocket 1502 can include zipper 1520, and opening 1522. Pocket 1504 can have channel 1524 for enclosing a wire connecting a thermistor to the electronics and/or a wire connecting a GSR sensor to the electronics. Channel 1524 can ensure that the sensors are positioned at an optimal location on the body for sensing.

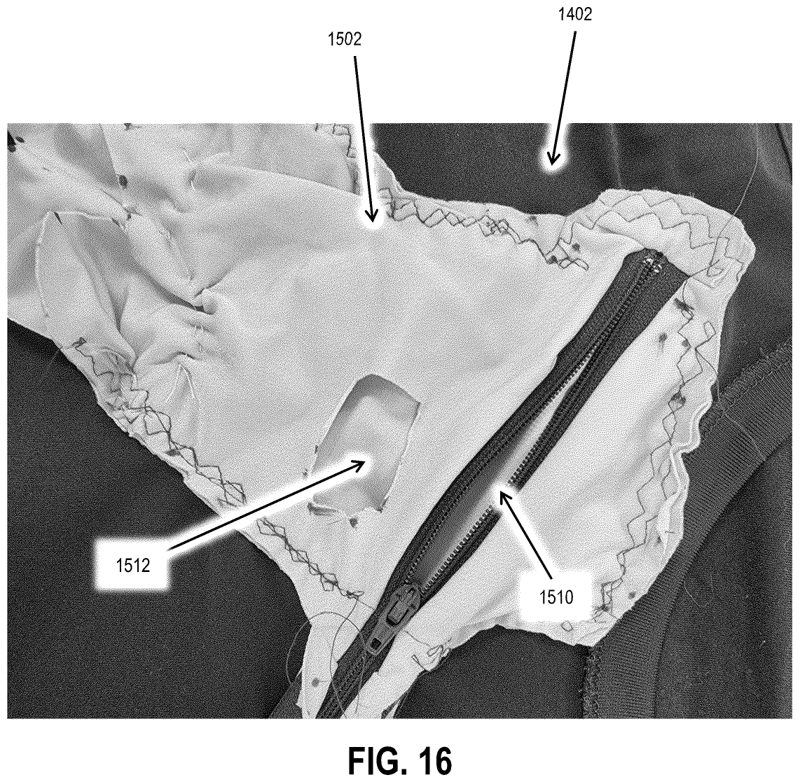

FIG. 16 shows a close up of a portion of pocket 1502. Pocket 1502 is enclosed by zipper 1510 so that the sensor package can be correctly inserted with ease and zipper 1510 was the least obstructive and uncomfortable fastener that was still sturdy enough to be reliable. Pocket 1504 can have zipper 1520 for similar reasons.

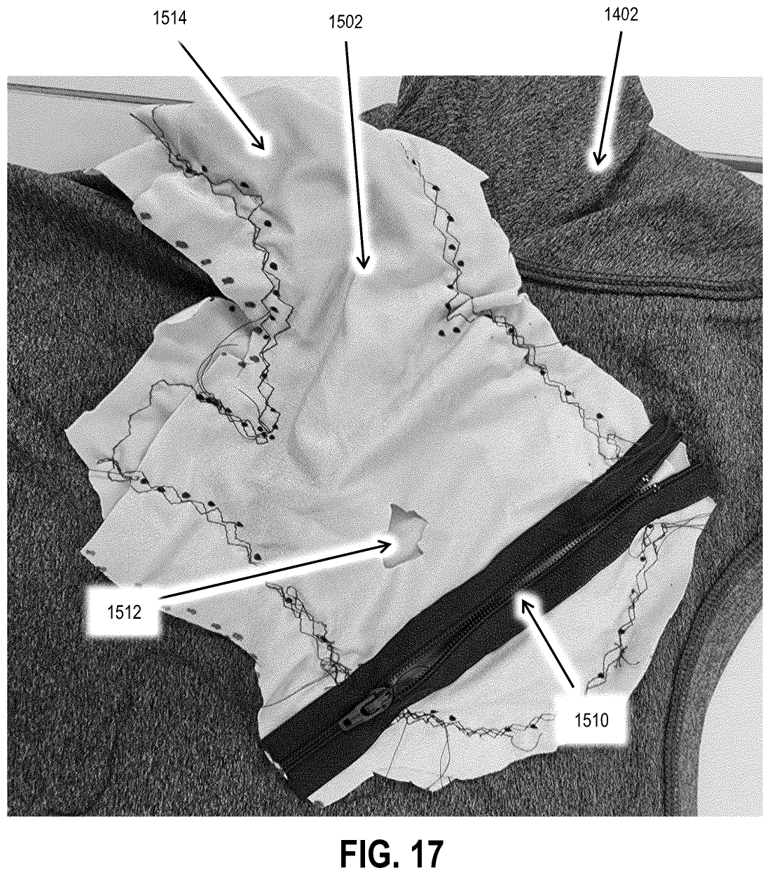

FIG. 17 shows another close up of a portion of pocket 1502. The shape of pocket 1502 intentionally packs most of the sensors and hardware into a large section, with channel 1514 (branch) extending into the armpit area in order to allow the thermistor to be in the correct position without getting bunched up or wrapped around other pieces of machinery or electronics. Pocket 1504 can have a similar shape and channel 1524 for similar reasons.

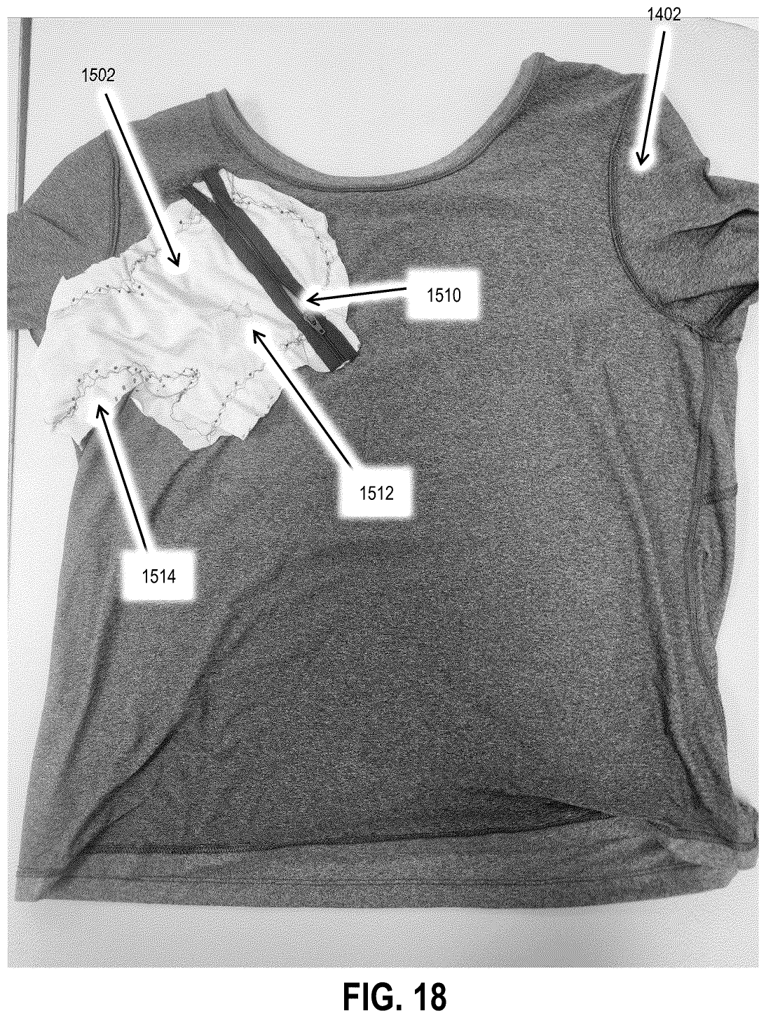

FIG. 18 illustrates that pocket 1502 is on the left front side of exemplary garment 1402. The blood oxygen monitor, which collects data through light through opening 1512, works most accurately as close to the heart as possible. In addition, the position of pocket 1502 accommodates the thermistor and/or GSR sensor reaching under the armpit through channel 1514 while keeping all wires as short as possible for safety, comfort, and functionality.

In some embodiments, the designs for the women's shirt has a larger difference in the bust, waist, and hip measurements compared to the men's shirt in order to make both shirts as form-fitting as possible.

In some embodiments, electronics are more easily secured on the garment through using more flexible materials for the pocket and the zipper orientation. Rather than using multiple layers stitched together, the fabric forms a uniform whole pocket. Instead of the zipper stretching the longer way of the pocket, the zipper is oriented across the shorter way of the pocket to secure the electronics with limited space to move. Lighter plastic waterproof film is used to enclose the electronics for flexibility and easy waterproofing.

### Variations on the Electronics System and Data Processing

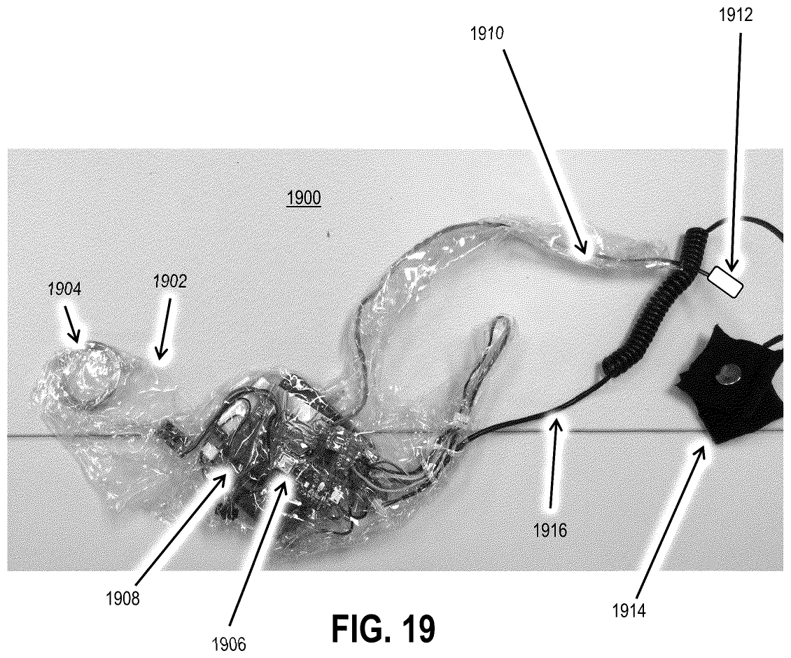

FIG. 19 is a photograph of exemplary electronics system 1900, according to some aspects of the disclosure. Exemplary electronics system 1900 includes one or more of: electronics package 1908 (e.g., having processor, circuits, printed circuit board, etc.), one or more sensors 1906 provided with electronics package 1908 (e.g., heart rate and SpO2 sensor), wireless charging coil 1904, wire 1910, thermistor 1912 coupled via wire 1910 to electronics package 1908, wire 1916, and moisture sensor 1914 coupled via wire 1916 to electronics package 1908.

At least a portion of exemplary electronics system 1900 is enclosed with waterproof film 1902 except for the thermistor 1912 and/or moisture sensor 1914 to ensure that the hardware is protected. Moisture sensor 1914 can include one or more electrodes, where an electrode can have a moisture-absorbing fabric surrounding the electrode. Wire 1910 and/or wire 1916 can have a bungee cord or stretchy coiled cord section for flexibility and comfort.

In some embodiments, exemplary electronics system 1900 includes battery protection using a load switch to prevent a battery from getting fried during charging.

In some embodiments, onboard sensors of exemplary electronics system 1900 can include one or more of: an accelerometer, gyroscope, and barometric pressure sensor. The accelerometer and/or the gyroscope can detect if SAR personnel has fallen, or is lying on the ground. The accelerometer and/or the gyroscope can detect if SAR personnel is walking or running. Barometric pressure sensor can determine elevation to account for altitude in health status calculations. Along with vital signs monitoring sensors, these sensors can offer further insights into what and how the SAR personnel is doing.

In some embodiments, sensors of exemplary electronics system 1900 can include a moisture sensor (e.g., Grove moisture sensor) for moisture and hydration level detection. The Grove moisture sensor can be adapted to measure sweat levels by utilizing its electrical conductivity mechanism. When the sensor is placed in contact with skin, it detects the moisture present in sweat through the varying resistance between its electrodes. Sweat, being conductive due to its water and electrolyte content, allows the sensor to generate a signal that reflects hydration and perspiration levels. This signal can be read by a microcontroller or integrated into wearable devices for applications such as fitness monitoring or health diagnostics to track sweat patterns and hydration status.

In some embodiments, moisture sensor data correlated with hydration is aggregated with data from other sensors to offer an assessment. An exemplary logic table is as follows:

| GSR (Skin | | Core | Blood | Result (Health | |
|---|---|---|---|---|---|
| Conduc- | Heart Rate | Tempera- | Oxygen | Status | Color/Visual |
| tance) Level | Level | ture Level | Level | Category) | Indicator |
| 0.5-3 micro | 60-100 bpm | 96-98 F. | 95-100% | Stable (resting) | GREEN |
| Siemens | Rest | | | | |
| | 100-160 | | | | |
| | Active | | | | |
| Low | High | High | Slightly | Possible | YELLOW |
| | | | Low | dehydration, | |
| | | | | maybe | |
| | | | | hypoxia | |
| Stable | Moderately | Stable | Stable | Normal | GREEN |
| | High | | | exertion in | |
| | | | | SAR scenario | |
| High | Moderately | Stable | Stable | Possible | YELLOW |
| | High | | | stress | |
| | | | | response | |
| Low | Stable | High | Stable | Early | YELLOW |
| | | | | dehydration | |
| Low | Stable | Stable | Low | Dehydration, | RED |
| | | | | low oxygen | |
| | | | | levels | |
| High | Low | High | Low | Severe | RED |
| | | | | dehydration | |

The rows represent different predetermined levels for different sensor data. The sensor data is evaluated to determine if the data matches the levels, and the result

[symbol]

In some embodiments, the processor of exemplary electronics system 1900 sends sensor data through a Blues wireless notecard. The sensor data can be sent as a JavaScript Object Notation packet to the backend in a separate “/data” pipeline. The backend can run the health data (heart rate, SpO2, temperature, moisture, etc.) through an algorithm to determine the overall health of the user (e.g., signified by red, yellow, and green). The backend then sends a request to the frontend through an API to change the marker and trail color representing the wearer.

In some embodiments, the backend can perform health status calculations at multiple time intervals and check for large deviations between previous values and the current recorded value. Anomalous deviations are removed and not transmitted to the frontend. Noise in the data can be filtered out and not transmitted to the frontend.

In some embodiments, the backend can track and analyze a wearer's baseline vitals and use the new recorded vitals to check for deviations from baseline. Ability to determine a wearer's particular baseline can be helpful as varied people have different types of body types and vitals.

In some embodiments, exemplary electronics system 1900 can include a power indicator comprising a light-emitting diode.

In some embodiments, exemplary electronics system 1900 can include a low power detection system to shut down the system if the battery power is too low or voltage supply is too low. The shutting down of the system can prevent erroneous or bad data from being transmitted to the backend, which can cause false alarms.

### Variations and Other Notes

Although the operations of the example methods shown in and described with reference to the FIGS. are illustrated as occurring once each and in a particular order, it will be recognized that the operations may be performed in any suitable order and repeated as desired. Additionally, one or more operations may be performed in parallel. Furthermore, the operations illustrated in the FIGS. may be combined or may include more or fewer details than described.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize. These modifications may be made to the disclosure in light of the above detailed description.

For purposes of explanation, specific numbers, materials and configurations are set forth in order to provide a thorough understanding of the illustrative implementations. However, it will be apparent to one skilled in the art that the present disclosure may be practiced without the specific details and/or that the present disclosure may be practiced with only some of the described aspects. In other instances, well known features are omitted or simplified in order not to obscure the illustrative implementations.

Further, references are made to the accompanying drawings that form a part hereof, and in which are shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the disclosed subject matter. However, the order of description should not be construed as to imply that these operations are necessarily order dependent. In particular, these operations may not be performed in the order of presentation. Operations described may be performed in a different order from the described embodiment. Various additional operations may be performed or described operations may be omitted in additional embodiments.

For the purposes of the present disclosure, the phrase “A or B” or the phrase “A and/or B” means (A), (B), or (A and B). For the purposes of the present disclosure, the phrase “A, B, or C” or the phrase “A, B, and/or C” means (A), (B), (C), (A and B), (A and C), (B and C), or (A, B, and C). The term “between,” when used with reference to measurement ranges, is inclusive of the ends of the measurement ranges.

The description uses the phrases “in an embodiment” or “in embodiments,” which may each refer to one or more of the same or different embodiments. The terms “comprising,” “including,” “having,” and the like, as used with respect to embodiments of the present disclosure, are synonymous. The disclosure may use perspective-based descriptions such as “above,” “below,” “top,” “bottom,” and “side” to explain various features of the drawings, but these terms are simply for ease of discussion, and do not imply a desired or required orientation. The accompanying drawings are not necessarily drawn to scale. Unless otherwise specified, the use of the ordinal adjectives “first,” “second,” and “third,” etc., to describe a common object, merely indicates that different instances of like objects are being referred to and are not intended to imply that the objects so described must be in a given sequence, either temporally, spatially, in ranking or in any other manner.

In the following detailed description, various aspects of the illustrative implementations will be described using terms commonly employed by those skilled in the art to convey the substance of their work to others skilled in the art.

The terms “substantially,” “close,” “approximately,” “near,” and “about,” generally refer to being within +/−20% of a target value as described herein or as known in the art. Similarly, terms indicating orientation of various elements, e.g., “coplanar,” “perpendicular,” “orthogonal,” “parallel,” or any other angle between the elements, generally refer to being within +/−5-20% of a target value as described herein or as known in the art.

In addition, the terms “comprise,” “comprising,” “include,” “including,” “have,” “having” or any other variation thereof, are intended to cover a non-exclusive inclusion. For example, a method, process, or device, that comprises a list of elements is not necessarily limited to only those elements but may include other elements not expressly listed or inherent to such method, process, or device. Also, the term “or” refers to an inclusive “or” and not to an exclusive “or.”

The systems, methods and devices of this disclosure each have several innovative aspects, no single one of which is solely responsible for all desirable attributes disclosed herein. Details of one or more implementations of the subject matter described in this specification are set forth in the description and the accompanying drawings.

What is claimed is:

1. A garment, comprising:
a pocket to securely hold an electronic sensing device against a skin of a wearer, the pocket including:
a main section to hold one or more onboard sensors and electronics of the electronic sensing device;
an opening in the main section to allow one of the one or more onboard sensors to detect skin measurements;
a closure mechanism in the main section to facilitate insertion and removal of the electronic sensing device; and
a channel extending from the main section to position at least one further sensor of the electronic sensing device at a location on the wearer, the at least one further sensor being coupled to the electronics via at least one or more wires.

2. The garment of claim 1, wherein a fabric of the pocket is stretchable or elastic.

3. The garment of claim 1, wherein the pocket is integrated into an inner side of the garment.

4. The garment of claim 1, wherein the one or more onboard sensors and the at least one further sensor include sensors for monitoring heart rate, blood oxygen saturation, and core temperature.

5. The garment of claim 1, wherein the electronic sensing device is at least partially enclosed by a water-resistant and transparent film.

6. The garment of claim 1, wherein the closure mechanism includes a zipper closure.

7. The garment of claim 1, wherein the one or more onboard sensors and the at least one further sensor include a thermistor to sense core temperature.

8. The garment of claim 1, wherein the one or more onboard sensors and the at least one further sensor include a moisture sensor to sense hydration.

9. The garment of claim 1, wherein the one or more onboard sensors and the at least one further sensor include an optical heart rate and SpO2 sensor.

10. The garment of claim 1, wherein the electronic sensing device comprises an inductive charging mechanism.

11. The garment of claim 1, wherein the channel positions the at least one further sensor near an armpit area of the wearer.

12. The garment of claim 1, wherein the garment includes a further pocket having a further main section, and a further channel.

13. A method for processing health status data, comprising:
receiving, by a backend, sensor data of a person from a plurality of sensors, the sensor data including heart rate, blood oxygen level, core temperature, skin conductance, and location;
evaluating the sensor data against predetermined levels to determine a health status category, the health status category selected from a plurality of predetermined health status categories;
determining, based on the health status category, a corresponding visual indicator selected from a green indicator, a yellow indicator, and a red indicator; and
displaying, by a frontend, the corresponding visual indicator on a map for real-time visual representation of the location and the health status category of the person.

14. The method of claim 13, further comprising filtering noise and/or anomalies from the sensor data prior to evaluating the sensor data against the predetermined levels.

15. The method of claim 13, wherein the predetermined levels are updated based on baseline data about the person.

16. The method of claim 13, wherein the green indicator corresponds to one or more categorized health categories, including stable resting state and normal exertion in a search and rescue scenario.

17. The method of claim 13, wherein the yellow indicator corresponds to one or more categorized health categories, including possible dehydration, stress response, and hypoxia.

18. The method of claim 13, wherein the red indicator corresponds to one or more categorized health categories, including severe hydration or low oxygen levels.

19. The method of claim 13, wherein evaluating the sensor data comprises selecting severe dehydration as the health status category based on detecting high skin conductance, low heart rate, high core temperature, and low blood oxygen level.

20. The method of claim 13, wherein evaluating the sensor data comprises selecting possible stress response as the health status category based on detecting high skin conductance, moderately high heart rate, stable core temperature, and stable blood oxygen level.
